(19) United States
(12) Patent Application Publication
(10) Pub. No.: US 2024/0307190 A1
(43) Pub. Date: Sep. 19, 2024

(54) INTERSOMATIC DEVICE

(71) Applicant: EMMEMME S.R.L.S., Rome (IT)

(72) Inventors: Antonino RACO, Rome (IT); Stefano CASELLI, Rome (IT); Massimo MISCUSI, Rome (IT); Claudio TRAVERSO, Rome (IT)

(21) Appl. No.: 18/280,269

(22) PCT Filed: Jan. 7, 2022
(86) PCT No.: PCT/EP2022/000006

§ 371 Date: Sep. 5, 2023

(30) Foreign Application Priority Data
Jan. 18, 2021 (IT) 102020000028514

(51) Int. Cl. A61F 2/44; A61F 2/30

(52) U.S. Cl. CPC A61F 2/446; A61F 2002/30387

(57) ABSTRACT

An intersomatic device (10), in particular for intersomatic vertebral stabilization, comprises at least two bodies (20, 30) that are reciprocally slidable in one another and cooperate so as to grow in volume in the intersomatic space until they cover an upper area of the receiving vertebral plates. An adjusting member (40) interacts with said bodies (20, 30) to define a first “compact” condition in which the device (10) and a second “extended” configuration, i.e. open and greater in volume, wherein the teeth of the first element (20) are moved and interfere minimally with those of the second element (30).

### TECHNICAL FIELD

The invention relates to an expandable intersomatic device that is used for intersomatic operations to the vertebral column and belongs to the field of medical appliances. The invention also relates to the use of said device to actuate the stabilization method.

### BACKGROUND ART

The main cause of vertebral pathologies is the degeneration of intervertebral discs.

The diminished ability of the intervertebral disc to perform its tasks often entails the need to intervene thereupon with the resulting possible implication of damage to the motion segment of the vertebral column.

Of the surgical treatments for treating this degeneration, the reference method today is the vertebral stabilization procedure with intersomatic arthrodesisa, an intervention that aims to re-establish the correct distance between two or more deteriorated intervertebral discs that are no longer able to perform their function.

Intersomatic stabilization is today obtained with different surgical methods depending on the anatomical district affected and the position of the main cause of the pain. The devices that are placed in the intersomatic space are cages of different materials and shapes.

The main drawback of the cages lies in the fact that they can act only as spacers or as spacers/containers of a substance that is suitable for biological stabilization of the segment subjected to the treatment.

It should also be said that for some surgical procedures, devices are used that are able to increase the height dimensions thereof, because the cages are expandable.

### DISCLOSURE OF INVENTION

In order to optimize the area of contact between the device and the receiving anatomical site, the invention relates to a device that, maintaining the requirements of anatomical and mechanical appropriacy, could increase in volume in the intersomatic space until it covers an upper area of the receiving vertebral plates.

In addition, expansion can occur on the transverse anatomical plane, a peculiarity that is not available in known devices.

In greater detail, the device can expand through the movement of components thereof that can slide reciprocally and move away from one another to create greater cover of the intersomatic space.

Owing to said feature of the invention, the operators will be allowed to perform surgical tasks that are minimally invasive that are otherwise necessary to be able to position intersomatic devices that have to cover a greater area of the vertebral plates.

Another peculiarity is that the device permits extension on the coronal plane to be able to possibly obtain also greater filling of the intervention space.

### BRIEF DESCRIPTION OF THE FIGURES

The features of the invention in question will be clear from the following description of preferred embodiments in accordance with what is proposed in the claims and with the help of the enclosed set of drawings, in which:

FIG. 1 illustrates the device of the object of the invention in a closed, i.e. compact configuration;

FIG. 2 illustrates the device of the object of the invention in an expanded configuration;

FIG. 3 illustrates two views of the device that is the object of the invention.

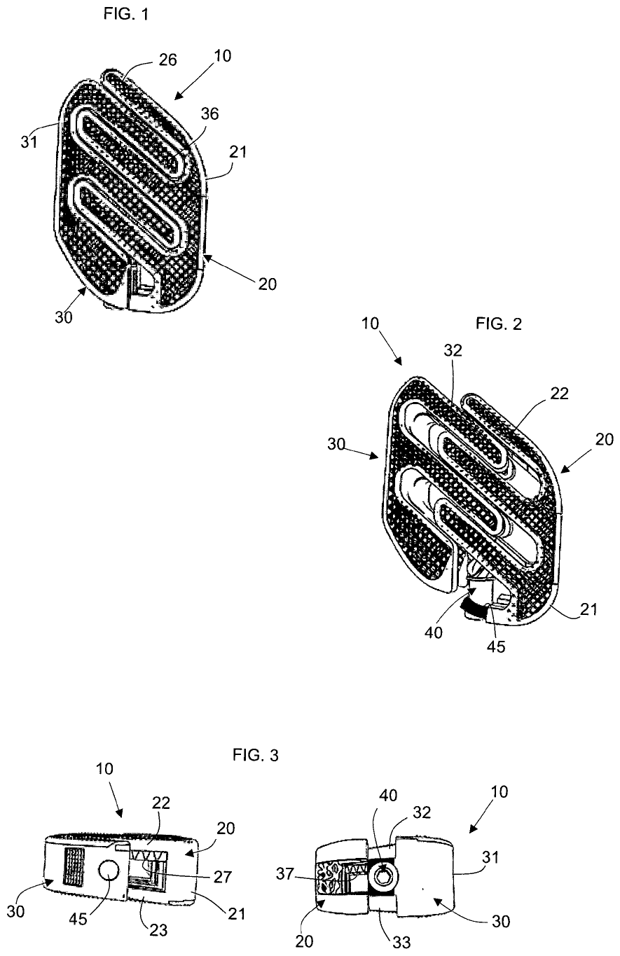

### BEST MODE FOR CARRYING OUT THE INVENTION

With reference to the figures shown, overall 10 indicates an expandable intersomatic device consisting of two bodies 20 and 30 that are reciprocally slidable in one another so that the device, whilst maintaining the requirements of anatomical and mechanical appropriacy, can at the same time be adjusted so as to increase in volume in the intersomatic space until it covers an upper area of the receiving vertebral plates. Each of said elements 20 and 30 comprise, laterally, a respective base 21, 31 from which a plurality of elements or teeth 22, 23 and 32, 33 depart and converge substantially perpendicular.

In greater detail, both the teeth 22, 23 and those indicated by 32, 33 that lead away from the ends of the respective end portions of the side bases 21, 31 and, overall, the bodies 20 and 30 form two combs.

Said bodies or combs 20, 30 are configured to interact together and cooperate together so as to define, by an adjusting member described below:
a first “compact” or closed condition in which the device 10 has the teeth of the first element 20 between those of the second element 30, or vice versa, and
a second “extended” configuration, i.e. open and greater in volume, in which the teeth of the first element 20 are moved and interfere minimally with those of the second element 30, i.e. interfering only with the respective ends 26, 36.

The adjustment between the two configurations occurs by means of an adjusting member indicated overall by 40.

Said adjusting member 40 is housed inside the space between the teeth of the combs 20, 30, being retained in respective seats 45 obtained in the bases 21, 31 and, overall, is arranged perpendicularly to the direction of the teeth 22, 23, 32, 33.

The adjusting member 40 is, preferably, a worm gear that engages a respective rack 27, 37 obtained on the inner faces of the teeth 22, 23, 32, 33. The teeth are preferably arranged in a direction that is substantially oblique to the arrangement of the member 40 and of the side bases 21, 31, and the rack is a rack with teeth that are oblique by the same angle of tilt as the teeth.

In this manner, rotation of the member 40 around its axis induces, by an oblique rack, a sliding action away from or towards the teeth 22, 23, 32, 33 of the two bodies 20 and 30 of the device 10 so as to be able to operate in the two “compact” and “extended” limit configurations indicated above.

The method for intersomatic vertebral stabilization thus uses at least one device 10 according to what has been described, so that both the corresponding method and the use thereof are protected.

1. Device (10), in particular for intersomatic vertebral stabilization, characterized in that it comprises at least two bodies (20, 30) that are reciprocally slidable in one another and cooperating so as to grow in volume in the intersomatic space until they cover an area that is greater than the receiving vertebral plates; and in which an adjusting member (40) interacts with said bodies (20, 30) to define:
A. a first “compact” or closed condition in which the device
(10) has the teeth of the first element (20) between those of the second element (30), or vice versa, and
B. a second “extended” configuration, i.e. open and greater in volume, in which the teeth of the first element (20) are moved and interfere minimally with those of the second element (30).

2. Device (10) according to claim 1, wherein
A. said elements (20, 30) comprise, laterally a respective base (21, 31) from which a plurality of elements or teeth (22, 23, 32, 33) depart or converge,
B. said adjusting member (40) is housed inside the space between said teeth (22, 23, 32, 33) and is retained in respective seats (45) obtained in the bases (21, 31).

3. Device (10), according to claims 1 and 2, wherein the adjusting member 40 is a screw that engages a respective rack (27, 37) obtained on the inner faces of at least two teeth (22, 23, 32,

4. Device (10), according to claims 1 and 2, wherein the teeth are preferably arranged in a direction that is substantially oblique to the arrangement of the member 40 and of the side bases 21, 31, and the rack is a rack with teeth that are oblique by the same angle of tilt as the teeth.

5. Method for intersomatic vertebral stabilization, characterized in that it provides for the use of at least one device (10) according to at least one of claims 1 to 4.

6. Use of a device (10) according to at least one of claims 10 to 4 to actuate the intersomatic vertebral stabilization.
